(19) United States
(12) Patent Application Publication
(10) Pub. No.: US 2024/0165398 A1
(43) Pub. Date: May 23, 2024

(54) MODULAR NEUROMODULATION ASSEMBLY

(71) Applicant: MALINDA HANSEN, FORT WORTH, TX (US)

(72) Inventor: MALINDA HANSEN, FORT WORTH, TX (US)

(21) Appl. No.: 18/057,755

(22) Filed: Nov. 21, 2022

(51) Int. Cl. A61N 1/04; A61N 1/36

(52) U.S. Cl. CPC A61N 1/0456; A61N 1/0488; A61N 1/36034; A61N 2001/083

(57) ABSTRACT

A modular neuromodulation assembly combines the ability to adjust both amplitude and frequencies for the benefit and improved outcomes of injuries and impairments in the arena of musculoskeletal rehabilitation and neural/motor patterning. The assembly includes a rechargeable power supply and a control board. An electric motor is used to regulate the amplitude and frequency of operation of the electric motor. An effector rod is selectively coupled to the electric motor and configured to extend the vibratory effects of the motor to the patient. The effector rod includes an effector tip coupled to a distal end of the effector rod. Operation of the electric motor induces a linear motion through the effector rod at a desired frequency and amplitude.

### BACKGROUND OF THE INVENTION

### 1. Field of the Invention

The present application relates to a neuromodulatory device, and more particularly to a modular neuromodulation device that is adjustable in frequency and amplitude.

### 2. Description of Related Art

Vibration has long been recognized as a useful modality for the human body since the 1880s when the French neurologist Jean-Martin Charcot noticed that patients suffering from Parkinson's experienced a reduction in their resting tremor after a train carriage ride or after horseback riding. Since then, a myriad of devices and inventions have been made and used. Specifically, the use of vibration in a rehabilitation setting has been more widely researched over the past 10-15 years showing benefit to low amplitude, high frequency vibrations.

Currently there are large hand-held massage guns that introduce vibration with very large amplitudes, and there are custom made vibratory devices for the purposes of research in the rehabilitation and functional setting. However, there are currently no devices on the market that can adjust both amplitude and frequency, which are essential for adjusting the neuromodulation response during rehabilitation.

Although strides have been made, shortcomings remain. It is desired that an assembly be provided that combines the ability to adjust both amplitude and frequencies for the benefit and improved outcomes of injuries and impairments in the arena of musculoskeletal rehabilitation and neural/motor patterning.

### BRIEF SUMMARY OF THE INVENTION

It is an object of the present application to provide an assembly configured to utilize variable amplitudes and frequencies for neuromodulation and increased efficacy of motor patterning. It is a further object of the present application that the assembly be capable of being handheld and neatly compact to allow a user to deliver the variable amplitudes and frequencies.

The assembly is configured to utilize battery powered technology to facilitate operation of a vibrating shaft with changeable effectors. This allows the assembly to be portable and highly convenient to a user. The vibrating effectors of the assembly are placed against a target muscle or muscle-tendon junction. The shaft/effector vibrates rapidly (i.e. up to 120 Hz) to create a window of neuromodulation for the brain, spinal cord, and target muscles to create, reinforce, or strengthen a motor pattern. The vibration amplitude may be adjustable between 0.1 mm to 1 mm.

Ultimately the invention may take many embodiments. In these ways, the present invention overcomes the disadvantages inherent in the prior art. The more important features have thus been outlined in order that the more detailed description that follows may be better understood and to ensure that the present contribution to the art is appreciated. Additional features will be described hereinafter and will form the subject matter of the claims that follow.

Many objects of the present application will appear from the following description and appended claims, reference being made to the accompanying drawings forming a part of this specification wherein like reference characters designate corresponding parts in the several views.

Before explaining at least one embodiment of the present invention in detail, it is to be understood that the embodiments are not limited in its application to the details of construction and the arrangements of the components set forth in the following description or illustrated in the drawings. The embodiments are capable of being practiced and carried out in various ways. Also it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the various purposes of the present design. It is important, therefore, that the claims be regarded as including such equivalent constructions insofar as they do not depart from the spirit and scope of the present application.

### BRIEF DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 is a perspective view of a modular neuromodulation assembly according to an embodiment of the present application.

FIG. 2 is a partial exploded perspective view of the assembly of FIG. 1.

FIG. 3 is a longitudinal section view of the assembly of FIG. 1.

While the embodiments and method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DETAILED DESCRIPTION OF THE INVENTION

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms to describe a spatial relationship between various components or to describe the spatial orientation of aspects of such components should be understood to describe a relative relationship between the components or a spatial orientation of aspects of such components, respectively, as the embodiments described herein may be oriented in any desired direction.

The embodiments and method in accordance with the present application overcomes one or more of the above-discussed problems commonly associated with the prior art discussed previously. In particular, the modular neuromodulation assembly of the present application is configured to provide a portable and compact neuromodulation device capable of adjusting both the amplitude and frequency of treatment. The assembly and its associated method of use utilize vibratory stimulus with variable amplitude and variable frequency through a hand-held device with changeable effector tips for use against the skin of the user. The assembly tips are configured to contact a musculotendinous junction to create a window of neuromodulation for increased corticospinal tract activity and neuromuscular patterning and reciprocal inhibition. This results in a benefit and increased efficacy of musculoskeletal rehabilitation. These and other unique features are discussed below and illustrated in the accompanying drawings.

The embodiments and method will be understood, both as to its structure and operation, from the accompanying drawings, taken in conjunction with the accompanying description. Several embodiments of the assembly may be presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless otherwise described.

Referring now to the Figures wherein like reference characters identify corresponding or similar elements in form and function throughout the several views. The following Figures describe embodiments of the present application and its associated features. With reference now to the Figures, embodiments of the present application are herein described. It should be noted that the articles “a”, “an”, and “the”, as used in this specification, include plural referents unless the content clearly dictates otherwise.

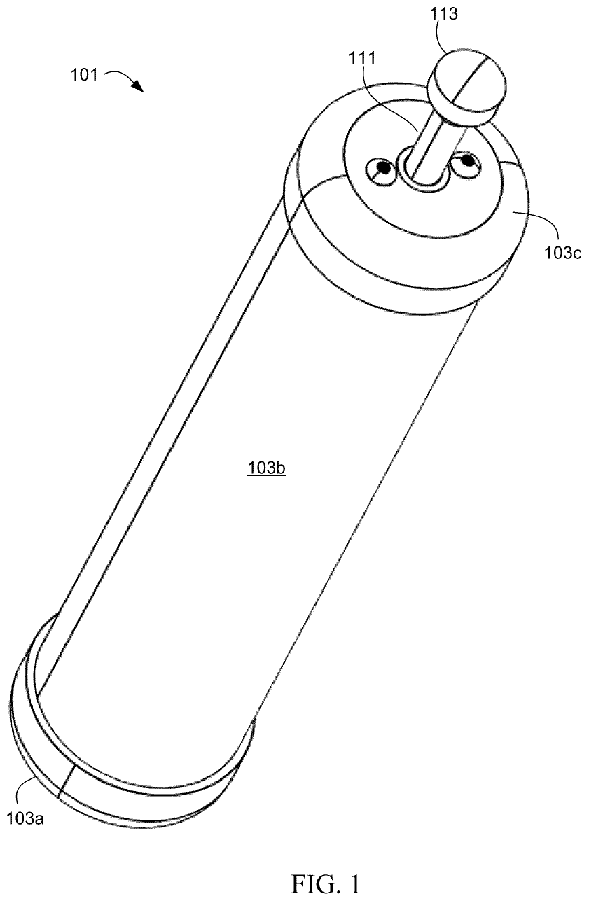

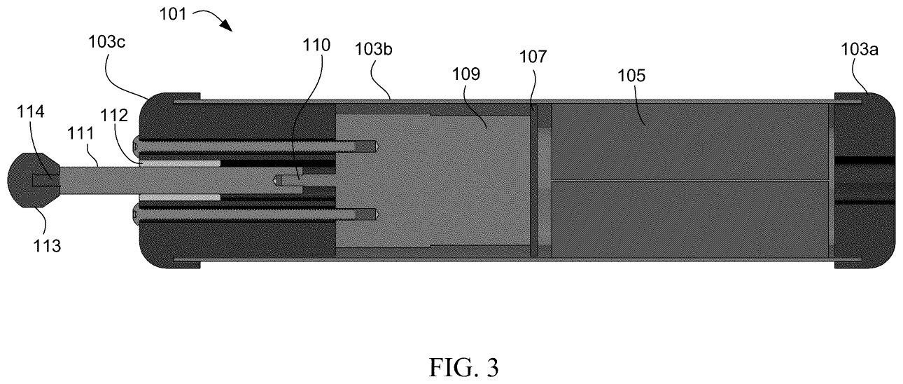

Referring now to FIGS. 1-3 in the drawings, a modular neuromodulation assembly 101 is illustrated. Assembly 101 is configured to induce a repeated linear movement against the surface of the skin of a user at one or more selected frequencies and/or amplitudes. The assembly includes a housing 103a-103c consisting of one or more parts that is configured to enclose one or more of the working components of assembly 101. Assembly 101 may be used as part of a rehabilitation treatment for a user as a result of multiple illnesses or conditions.

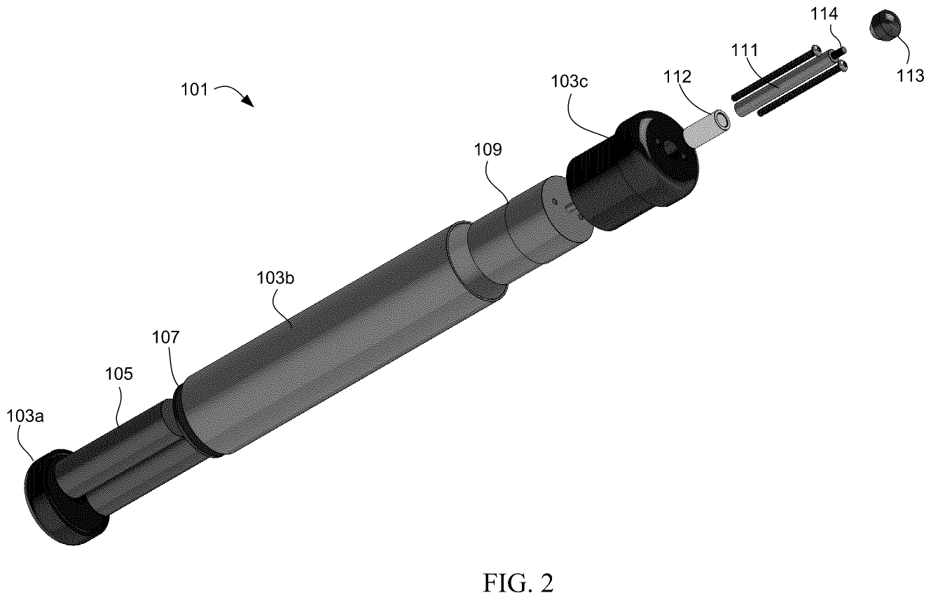

As seen in particular with FIG. 2 of the drawings, an exploded view of assembly 101 is provided wherein the internal components to housing 103a-103c are shown in greater detail. Assembly 101 further includes a power supply 105, a control board 107, an electric motor 109, an effector rod 111 with a corresponding effector tip 113.

Power supply 105 is configured to provide electrical power to the various components of assembly 101. Power supply 105 is in communication with control board 107 and electric motor 109. It is understood that various methods of powering assembly 101 may be provided or used. In this embodiment, power supply 105 is rechargeable batteries that service to provide portability and avoid the need for a continual plugged in source of power. Power supply 105 may be removed with the separation of housing member 103a from housing member 103b.

Control board 107 is configured to receive power from power supply 105. Board 107 is configured to monitor and regulate operation of electric motor 109. Board 107 is programmed to receive and input data from motor 109 as well as transmit one or more command data so as to adjust performance of motor 109. Board 107 may be programmed in accordance with the needs of the user wherein a user may optionally select modes or provide general inputs into performance parameters for board 107 in operating the assembly 101 for treatment. The user control inputs are optional and are not required.

Electric motor 109 is configured to communicate with both power supply 105 and board 107. Electric motor 109 is configured to receive an electric signal and generate an oscillatory movement. Motor 109 includes a shaft 110 that translates along an axis in a forward and rearward motion. Although not limited to this embodiment, electric motor 109 may be a voice coil actuator. A voice coil actuator is a type of direct drive motor configured to selectively actuate one or more effector rod 111 via shaft 110. The voice coil converts electrical signals directly into a linear magnetic force. The linear magnetic force is applied to the effector rod 111. Use of a voice coil actuator is preferred as it may provide higher quality of motion compared to conventional DC motors.

Control board 107 is configured to monitor the performance of motor 109. Such parameters that may be monitored may include, the pressure applied to the shaft 110, the speed of shaft 110, the position of shaft 110, the length of translation of shaft 110, the amount of power used in operation, and so forth. For example, board 107 may be set to include a pressure threshold which is a maximum level of pressure that may be applied to the end of motor 109 through the effector rod 111. An exemplary pressure level may be 2.5 pounds of pressure. Upon reaching the pressure threshold, board 107 may cease vibration functions via motor 109. This may be thought of as a safety feature to avoid a user applying too much pressure to a surface. This pressure threshold may be set to any predetermined level in accordance with design constraints. Knowing the position of shaft 110 allows board 107 to adjust the power output to maintain the desired trajectory. Board uses closed loop control with respect to the axial position of the voice coil actuator to deliver controllable and repeatable therapy.

Effector rod 111 is coupled to a portion of shaft 110. Rod 111 passes through an aperture in housing member 103c and is axially aligned with shaft 110. Movement of shaft 110 correspondingly translates rod 111 in a similar manner of motion. Movement of shaft 110 is made in small and changeable amplitudes and frequencies. Rod 111 extends the motion of motor 109 outside of the housing to reduce obstructions for the user from the size of the housing. Rod 111 may be made from a rigid or flexible polymer with a modular interface to change transmission force on user. In one embodiment, rod 111 makes use of ceramic coated aluminum that interfaces with wear-resistant PEEK to protect motor from off-axis loading and reduce friction.

A bearing 112 may be used to separate effector rod 111 from housing member 103c so as to reduce resistance and control orientation of rod 111. As seen in FIG. 2, one or more fasteners may be used to secure housing member 103c to housing member 103b and or motor 109. Housing member 103c may be selectively removed as needed.

A distal end 114 of effector rod 111 includes an attachment member which is used to couple to effector tip 113. Effector tip 113 is configured to engage a surface on the user for treatment. The motion of effector rod 111 is transferred to the patient/user via effector tip 113. Effector tip 113 is configured to be interchangeable. The size and material composition of tip 113 can be varied from one component to another. For example, tip 113 may be a foam tip, relatively soft, that interfaces with the user. In another example, the tip 113 may be harder than foam but the size is modified to correlate with the area of treatment on the user.

In use, the user obtains assembly 101 and selects the effector tip appropriate for the treatment and treatment location on the user. Assembly 101 is activated and subsequently located against a surface of the user for treatment. Board 107 and the user may regulate the amplitude and frequency of motion of the effector rod either before, during or after treatment. The amplitude and frequency may be adjusted automatically. Board 107 continues to monitor the pressure and other parameters of assembly 101. If, for example, the pressure threshold is reached, the power output may be restricted in some manner.

The particular embodiments disclosed above are illustrative only, as the application may be modified and practiced in different but equivalent manners apparent to those skilled in the art having the benefit of the teachings herein. It is therefore evident that the particular embodiments disclosed above may be altered or modified, and all such variations are considered within the scope and spirit of the application. Accordingly, the protection sought herein is as set forth in the description. It is apparent that an application with significant advantages has been described and illustrated. Although the present application is shown in a limited number of forms, it is not limited to just these forms, but is amenable to various changes and modifications without departing from the spirit thereof.

What is claimed is:

1. A neuromodulation assembly, comprising:
a power supply;
a control board in communication with the power supply;
an electric motor in communication with the control board, the control board configured to regulate the amplitude and frequency of operation of the electric motor;
an effector rod selectively coupled to the electric motor and configured to extend the vibratory effects of the motor; and
an effector tip coupled to a distal end of the effector rod;
wherein operation of the electric motor induces a linear motion through the effector rod at a desired frequency and amplitude.

2. The assembly of claim 1, wherein the effector tip is interchangeable.

3. The assembly of claim 1, wherein the effector tip is sized in accordance with selected treatment locations.

4. The assembly of claim 1, wherein the power supply is a rechargeable battery.

5. The assembly of claim 1, wherein the control board is configured to measure the position of a shaft in the electric motor so as to regulate the amplitude and frequency of motion.

6. The assembly of claim 1, wherein the electric motor is a voice coil actuator.

7. The assembly of claim 1, further comprising:
a housing configured to extend around the power supply, the control board, the electric motor, and a portion of the effector rod.

8. The assembly of claim 1, wherein the control board is configured to monitor the pressure applied to the effector rod during operation.

9. The assembly of claim 8, wherein the control board has a threshold of pressure in which it will cease vibration.

10. The assembly of claim 9, wherein the threshold of pressure is 2.5 pounds of pressure.

11. A method of neuromodulation treatment, comprising:
obtaining a neuromodulation assembly of claim 1;
selecting the effector tip in accordance with an area of a user to be in contact with the neuromodulation assembly;
activating the neuromodulation assembly;
locating the neuromodulation assembly against a surface of the user; and
regulating the amplitude and frequency of motion of the effector rod.

12. The method of claim 11, further comprising:
automatically adjusting the power output of the electric motor.

13. The method of claim 11, further comprising:
monitoring a pressure applied to the user via the effector rod.

14. The method of claim 13, further comprising:
automatically adjusting the power output upon reaching a pressure threshold.
